(19) United States
(12) Patent Application Publication
(10) Pub. No.: US 2020/0211708 A1
(43) Pub. Date: Jul. 2, 2020

(54) SUSTAINABLE TYPE 2 DIABETES PREVENTION MODULE

(71) Applicants: Christine Tinio Geronimo-Button, Manorville, NY (US); Terry Michael Button, Manorville, NY (US); Ashish Verma, Pt. Jefferson, NY (US)

(72) Inventors: Christine Tinio Geronimo-Button, Manorville, NY (US); Terry Michael Button, Manorville, NY (US); Ashish Verma, Pt. Jefferson, NY (US)

(21) Appl. No.: 16/793,572

(22) Filed: Feb. 18, 2020

Related U.S. Application Data
(63) Continuation-in-part of application No. 14/189,546, filed on Feb. 25, 2014, now abandoned.
(60) Provisional application No. 61/768,826, filed on Feb. 25, 2013.

(51) Int. Cl. G16H 50/20; A61B 5/145; G16H 50/30; G16H 20/60; G16H 20/30; G16H 10/60

(52) U.S. Cl. CPC G16H 50/20; A61B 5/14532; G16H 10/60; G16H 20/60; G16H 20/30; G16H 50/30

(57) ABSTRACT

A method is disclosed for monitoring or delaying or preventing or combinations thereof, the onset of Type 2 diabetes or reducing blood sugar level to a normal range that includes interfacing, via a computer-based system, a user device with a processor in communication with one or more data bases; inputting at least one user profile into one or more of the data bases; inputting data into one or more data bases wherein the data relates to diabetes optimized dietetic or diabetes optimized exercise or weight or combinations for at least one user corresponding to the at least one user profile; and presenting to at least one user a summary of the data input into the one or more data bases wherein the data relates to diabetes optimized dietetic or diabetes optimized exercise or weight or combinations. A corresponding system and an article of manufacturing are also disclosed.

### CROSS-REFERENCE TO RELATED APPLICATIONS

This application is a Continuation-in-Part of U.S. patent application Ser. No. 14/189,546 filed Feb. 25, 2014, and claims priority to U.S. Patent Application 61/768,826. filed on Feb. 25, 2013, the contents of each of which are incorporated by reference.

### FIELD OF THE INVENTION

This invention relates to computer implemented systems and user interfaces useful in the treatment or prevention of diabetes (elevated blood sugar).

### BACKGROUND

Diabetes mellitus (DM), commonly known as diabetes, is a group of metabolic disorders characterized by a high blood sugar level over a prolonged period of time. If left untreated, diabetes can cause many complications including diabetic ketoacidosis, hyperosmolar hyperglycemic state, or death. Serious long-term complications include cardiovascular disease, stroke, chronic kidney disease, foot ulcers, damage to the nerves, and damage to the eyes.

This invention pertains to Type 2 diabetes, which begins with insulin resistance, a condition in which cells fail to respond to insulin properly. As the disease progresses, a lack of insulin may also develop. This form was previously referred to as “non-insulin-dependent diabetes mellitus” (NIDDM) or “adult-onset diabetes”. The most common cause is a combination of excessive body weight and insufficient exercise.

Prediabetes is glucose dysregulation defined by impaired fasting blood glucose levels (100-125 mg/dL) or 2-h plasma glucose test (140-199 mg/dL) or HbA1C values between 5.7% and 6.4% (1). Patients with prediabetes face an increased risk of developing diabetes and other chronic illnesses such as cardiovascular and renal disease. (2) An estimated 84.1 million people in the United States had prediabetes in 2015. The burden of prediabetes was estimated to be the highest among adults between the age groups of 45 and 64 years, as well as in racial and ethnic minorities. (3) The cost of prediabetes increased by 74% between 2007 and 2012 and is predictive of economic burden due to new future cases of diagnosed diabetes. (4)

The Finnish Diabetes Prevention Study (5) and the National Institutes of Health (NIH) Diabetes Prevention Program (DPP) (6) demonstrated that the onset of type II diabetes could be prevented or delayed through healthy eating, increased physical activity, and modest weight loss. Lifestyle modifications leading to 7% body weight loss and moderate physical activity of 150 min/week were found to delay the progression of prediabetes and reduce the risk of developing diabetes by 58%. (7) Johnson and Melton (8) studied perceived barriers to evidence-based health intervention programs and found that 25% of study participants deterred from attending classes due to time or schedule of class and 8% due to transportation. Smartphone usage has increased from 35% in 2011 to 77% in 2018. (9) Mobile-based health solutions could be utilized as an effective alternative solution to motivate the users to utilize the health coaching tool from their home and at the convenience of their schedule.

A recent meta-analysis concluded that smartphone-based health applications which included some form of personal contact had higher rates of weight reduction as compared to completely digital algorithms. (10) A multitude of modalities (11-14) have been tried to implement the lifestyle intervention programs and there is no clear evidence as to which model is more superior; however, there is significant evidence to show that digital modalities are a cost-effective measure with wider outreach. (15-18).

Fundamental damage caused by high blood sugar level for extended time is at the level of the microvascular. Apparently high glucose blood level impairs fatty acid synthesis and normal nitric oxide synthesis in endothelial cells. This leads to vessels that are leaky and more susceptible to infection and less able to facilitate repair. While the cause of diabetic neuropathy is not clear, it is likely the cause of a combination of factors: high blood glucose levels, abnormal blood fat levels, low insulin levels and neurovascular damage resulting in reduced oxygen and nutrients.

This fundamental damage can manifest over the long term for uncontrolled diabetes to cause severe medical consequence. Uncontrolled diabetes is a leading cause of blindness, end-stage renal disease and limb amputation. Diabetics have more than doubles the risk of heart disease as well as increased risk of stroke.

Glycated hemoglobin (HbA1c, hemoglobin A1C, or just A1C), is a form of hemoglobin (abbreviated Hb) that is chemically linked to a sugar. Most monosaccharides, including glucose, galactose and fructose, spontaneously (i.e. non-enzymatically) bind with hemoglobin, when present in the bloodstream of humans. The formation of the sugar-Hb linkage indicates the presence of excessive sugar in the bloodstream, often indicative of diabetes. A1C is of particular interest because it is easy to detect. The A1C blood test provides an index of average blood glucose for the previous three to four months. Normal A1C blood sugar levels are generally considered to be 5.6% or below. Prediabetic blood sugar level is considered to be 5.7% to 6.4%. A1C values of 6.5 or above indicate diabetes.

There are an estimated 79 million U.S. citizens with prediabetes or high blood sugar but not yet at the level to be classified as diabetes. If prediabetics do not make life style changes to control blood sugar, their condition can advance to the point that their blood sugar elevates such that they become diabetic.

A variety of electronic tools are found on the web and/or as products that are geared toward weight loss, dietary control and/or exercise motivation that could apparently be useful in diabetes prevention. It is important to note, however, that these tools as available have not adequately addressed the epidemic of prediabetes. The number of prediabetics has rocketed due to poor eating and under activity to the point that the direct medical financial threat of prediabetics becoming diabetics is nearly $3 trillion in the coming decade in the US alone.

### BRIEF SUMMARY OF THE INVENTION

In order to address the problem of diabetes and prediabetes, the instant invention provides computer-implemented systems, user interfaces, and methods to provide a virtual coaching component that delivers automated messages and advice to users in real-time, where the automated messages and advice are based on user input.

In one aspect, the embodiments of the present disclosure relate to a method for monitoring or delaying or preventing or combinations thereof, the onset of Type 2 diabetes or reducing blood sugar level to a normal range that addresses the foregoing problems. The method includes interfacing, via a computer-based system, a user device with a processor in communication with one or more data bases; inputting at least one user profile into one or more of the data bases; inputting data into one or more data bases wherein the data relates to diabetes optimized dietetic or diabetes optimized exercise or weight or combinations thereof of at least one user corresponding to the at least one user profile, and presenting to at least one user a summary of the data input into the one or more data bases wherein the data relates to diabetes optimized dietetic or diabetes optimized exercise or weight or combinations thereof.

Thus, in an embodiment, a system is provided having a memory that stores computer executable components. The system may also include a processor, operably coupled to the memory, and that executes computer executable components stored in the memory, wherein the computer executable components comprise components tracking exercise, food consumption, body weight, blood sugar measurements, and blood A1C data, or a combination thereof of a user diagnosed with diabetes or pre-diabetes. The system may also include a virtual coaching component that determines a set of optimized dietary or optimized exercise scores, or both, based on algorithms employing data on exercise, food consumption, body weight, blood sugar measurements, and blood A1C data, or a combination thereof of the user. The scores may be used to by the virtual coaching component to make real-time specific recommendations of diet, exercise, and lifestyle elements calculated to reduce blood sugar in the user.

In an embodiment, the real-time specific recommendations of diet, exercise, and lifestyle elements are presented on a user-interface on a smartphone or web app. In an embodiment, the real-time specific recommendations of diet, exercise, and lifestyle elements provided to the user at least three times per day. These may be at preset times, or spaced randomly during the day.

In an embodiment, a user interface is provided for a user diagnosed with diabetes or pre-diabetes. The user interface provides a means for the user to interact with computer application that includes a memory that stores computer executable components. The computer application may also include a processor, operably coupled to the memory, and that executes computer executable components stored in the memory, wherein the computer executable components comprise components tracking exercise, food consumption, body weight, blood sugar measurements, and blood A1C data, or a combination thereof of a user diagnosed with diabetes or pre-diabetes. The computer application may also include a virtual coaching component that determines a set of optimized dietary or optimized exercise scores, or both, based on algorithms employing data on exercise, food consumption, body weight, blood sugar measurements, and blood A1C data, or a combination thereof of the user. The scores may be used to by the virtual coaching component to make real-time specific recommendations of diet, exercise, and lifestyle elements calculated to reduce blood sugar in the user.

### BRIEF DESCRIPTION OF THE DRAWINGS

FIG. 1 is a schematic embodiment of the system and apparatus of the instant invention.

FIG. 2 is a schematic of components comprising the application memory 120.

FIG. 3 is a schematic of the operation of the virtual coach component.

FIG. 4A is a screenshot of a notification bar icon on a smartphone, signaling to the user that a notification message has been generated.

FIG. 4B is a screenshot of a message notification screen, with instructions.

FIG. 4C is a screenshot of a user input screen, in this case a dietary input.

FIG. 5 is a screenshot of options in the mobile app.

FIG. 6A is a daily summary report.

FIG. 6B is a weekly summary report.

FIG. 6C is a second page of the weekly summary.

### DETAILED DESCRIPTION

Type 2 diabetes is characterized by insulin resistance, which may be combined with relatively reduced insulin secretion. The defective responsiveness of body tissues to insulin is believed to involve the insulin receptor. However, the specific defects are not known. Diabetes mellitus cases due to a known defect are classified separately. Type 2 diabetes is the most common type of diabetes mellitus. Many people with type 2 diabetes have evidence of prediabetes (impaired fasting glucose and/or impaired glucose tolerance) before meeting the criteria for type 2 diabetes. The progression of prediabetes to overt type 2 diabetes can be slowed or reversed by lifestyle changes or medications that improve insulin sensitivity or reduce the liver's glucose production.

Type 2 diabetes is primarily due to lifestyle factors and genetics. A number of lifestyle factors are known to be important to the development of type 2 diabetes, including obesity (defined by a body mass index of greater than 30), lack of physical activity, poor diet, stress, and urbanization. Excess body fat is associated with 30% of cases in those of Chinese and Japanese descent, 60-80% of cases in those of European and African descent, and 100% of Pima Indians and Pacific Islanders.

Dietary factors also influence the risk of developing type 2 diabetes. Consumption of sugar-sweetened drinks in excess is associated with an increased risk. The type of fats in the diet is also important, with saturated fat and trans fats increasing the risk and polyunsaturated and monounsaturated fat decreasing the risk. Eating lots of white rice also may increase the risk of diabetes, whereas substitution of brown rice or other whole grains for white rice may lower the risk of diabetes. A lack of physical activity is believed to cause 7% of cases.

Given this spectrum of lifestyle factors implicated in Type 2 diabetes, others have attempted automated tools to prevent or reverse elevated blood sugar. See, for example, US 2002/0187463 A1. However, that invention lacks many of the features and benefits of the instant invention.

The failure of prior electronic tools to significantly reduce the onset of Type 2 diabetes is the result of a design that may not specifically address diabetes prevention. Tools required to prevent diabetes must contain an appropriate educational foundation, dietary direction and physical activity requirements. For example, vigorous exercise programs can actually increase blood sugar. Moderate exercise over a long period, however, can dramatically reduce blood sugar. Regarding diet, eating fewer meals to lose weight is typically ineffective. However, six small meals each day rich in carbohydrates may prevent ebbs and spikes in blood sugar.

The embodiments of the present disclosure are designed to provide an educational foundation and support tools in the form of an electronic diary and virtual coach to at least monitor or delay or prevent, or combinations thereof, the onset of Type 2 diabetes, if not convert the blood sugar level of prediabetics back to the normal range.

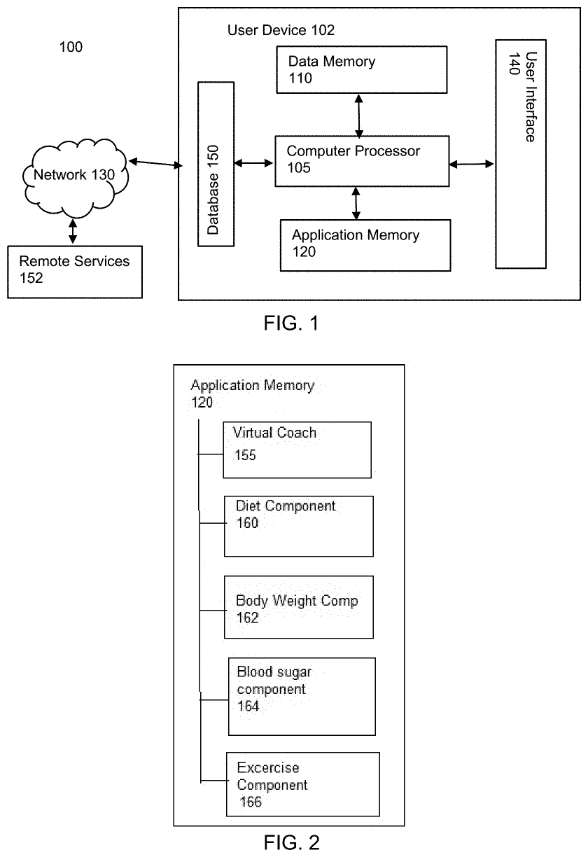

Referring to FIG. 1, an exemplary system, or article of manufacturing, 100 for monitoring or delaying or preventing the onset of Type 2 diabetes or reducing blood sugar to a normal level or combinations thereof, is disclosed. Hereinafter, reference to system 100 also includes reference to an article of manufacture. System 100 may comprise a user device 102. The user device may be a computational device, which may be a smartphone (for example, Android or iPhone device), a computer tablet, or some other computer. The user device will include several components, for example, a processor 105, data memory 110, application memory 120, user interface 140, and database 150. Additional components that may internal to the user device or external include a network 130, and remote services 152.

The processor 105 may be a computer processor, of which many variations are known in the art. The data memory 110 may be random access memory (RAM) and stores critical information temporarily. For example, user input data is entered into RAM and used interactively by the processor and applications running on the processor. Application memory 120 may be based on RAM or some other type of memory and in an embodiment, contains several components. (FIG. 2). For example, the Virtual Coach 155 may be one component. Other components may be the diet component 160, a body weight component 162, a blood sugar component 164, and an Exercise tracking component 166. This is a non-limiting list. Other components may be present also. Each of these components may include algorithms or programmatic steps that take user inputs (for example, from an interactive user interface), and provide outputs that may be deposited in the database 150 and/or displayed to the user via the user interface.

Some embodiments of this invention refer to a “diary,” which is permanent record of some kind, for example blood sugar tracked over a period of time, dietary records tracked over a period of time, etc. In an embodiment, the various data inputs and outputs from the components in the application memory 120 are stored permanently in the database 150. By “permanent,” it is meant that the database is non-volatile memory that persists even if the database is powered down. Thus, a “diary” may be user-friendly term for data stored in a database that can be retrieved at a later date. Data in the database may be transmitted to coaches, supervisors, or physicians. Data on the database 150 may also be used to generate weekly, monthly, or other time period reports. In an embodiment, the database 150 is part of the user device. In an alternative embodiment, the database is in a remote location and is accessed via the network.

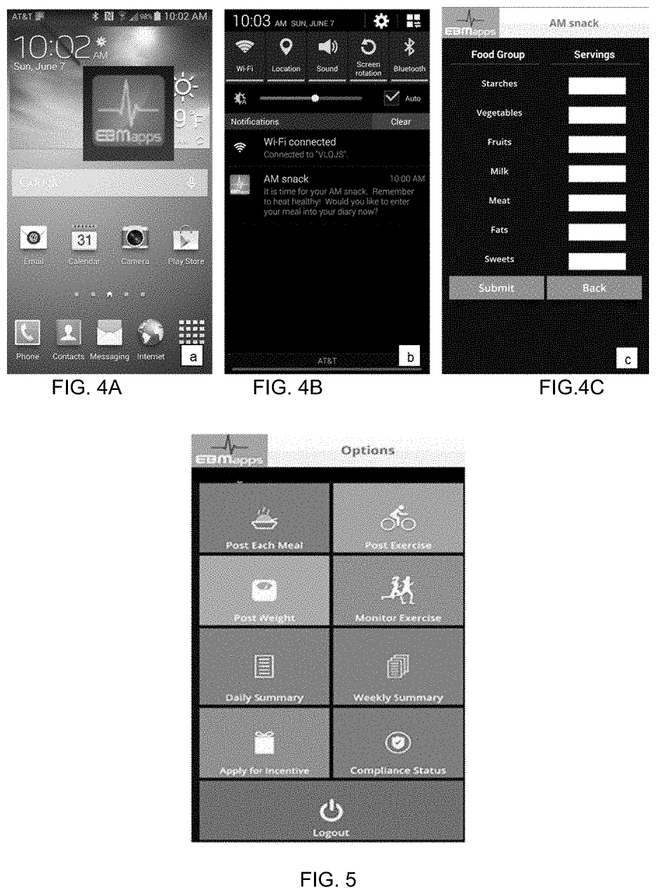

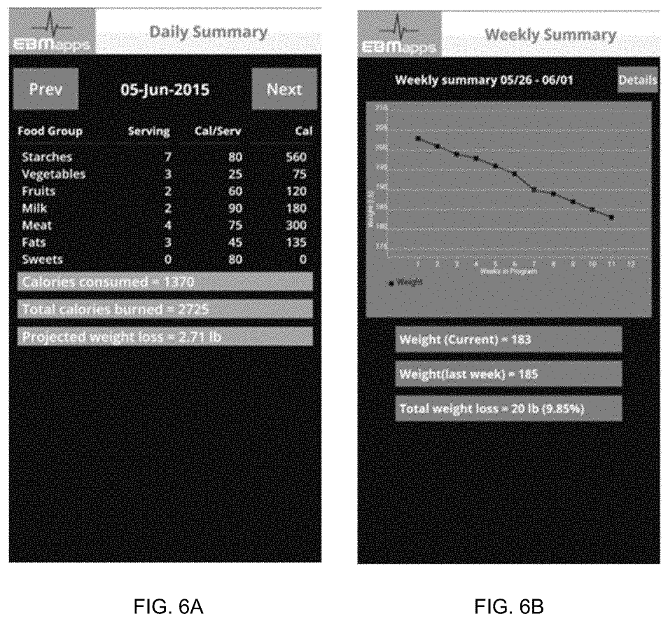

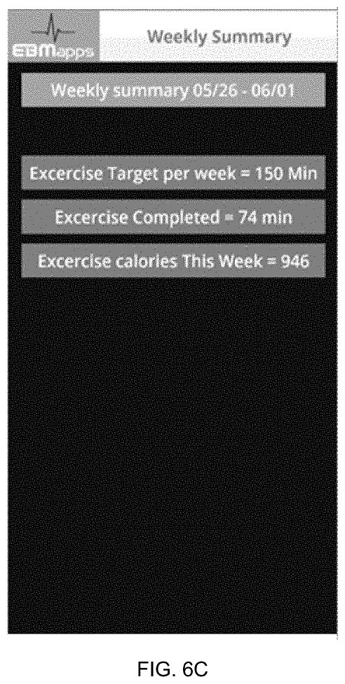

The user interface 140 may include a variety of screens for the output of data for presentation to users, for example as shown in FIGS. 6A-C. Other screens may be used for user input, for example FIG. 4C and FIG. 5, where users select options or enter data.

Network 130 may provide access to other services, for example for coaches, supervisors, or physicians. These are shown in FIG. 1 as remote services 152. T

As those skilled in the art will appreciate, a user device 102 may include an operating system (e.g., Windows, Linux, MacOS, iOS, Android, etc.) as well as various conventional support software and drivers typically associated with computers. A user device may implement security protocols such as Secure Sockets Layer (SSL) and Transport Layer Security (TLS). A user device may implement one or more application layer protocols, including, for example, http, https, ftp, and sftp. Transactions originating at a user device may pass through a firewall (not shown; see below) in order to prevent unauthorized access from users of other networks.

Network 130 may comprise any cloud, cloud computing system or electronic communications system or method which incorporates software and/or hardware components. Communication may be accomplished through any suitable communication channels, such as, for example, a telephone network, an extranet, an intranet, Internet, point of interaction device (point of sale device, personal digital assistant, smart phone, cellular phone (e.g., iPhone®, kiosk, etc.), online communications, satellite communications, off-line communications, wireless communications, transponder communications, local area network (LAN), wide area network (WAN), virtual private network (VPN), networked or linked devices, keyboard, mouse and/or any suitable communication or data input modality. Moreover, although network 130 may be described herein as being implemented with TCP/IP communications protocols, the network 130 may also be implemented using IPX, Appletalk, IP-6, NetBIOS, OSI, any tunneling protocol (e.g. IPsec, SSH), or any number of existing or future protocols. If the network 130 is a public network, such as the Internet, it may be advantageous to presume the network 130 to be insecure and open to eavesdroppers. Specific information related to the protocols, standards, and application software utilized in connection with the Internet is generally known to those skilled in the art and, as such, need not be detailed herein. In an embodiment, a network 130 may be excluded from system 100. More particularly, system 100 may comprise a mainframe system and/or a single distributed system

The various system components described herein may be independently, separately or collectively coupled to the network 130 via one or more data links including, for example, a connection to an Internet Service Provider (ISP) over a local loop as is typically used in connection with home or office networks, or public WiFi.

“Cloud” or “Cloud computing” includes a model for enabling convenient, on-demand network access to a shared pool of configurable computing resources (e.g., networks, servers, storage, applications, and services) that can be rapidly provisioned and released with minimal management effort or service provider interaction. Cloud computing may include location-independent computing, whereby shared servers provide resources, software, and data to computers and other devices on demand. Specific information related to the protocols, standards, and application software utilized in connection with cloud computing is generally known to those skilled in the art and, as such, need not be detailed herein. A database may comprise any type of hardware and/or software (e.g., a computer server) configured or configurable to host a database. Typically, such a server comprises a rack mountable server appliance running a suitable database server (e.g., SQL Server, an Oracle database, and the like), and/or one or more virtual machines. Accordingly, database may include its own processor and other computer components.

In an embodiment, the invention includes a virtual coach 155. The virtual coach is an artificial intelligence module, that takes a variety of inputs in real time and generates messages, suggestions, instructions, notifications, and reports for the user and coaches, supervisors, and physicians. In an embodiment, the virtual coach may be a neural network.

Various professional or trained staff may be involved in working with a user (i.e., a person diagnosed with pre-diabetes or diabetes). Staff may include physicians, supervisors, and coaches. “Physicians” refers here to persons with medical degrees, such as MD's (Medical Doctors) or DO's (Doctors of Osteopathy). “Supervisors” refers here to other trained and professional staff, such as physician's assistants and nurses, trained to work with persons with elevated blood sugar. “Coaches” may refer to other trained individuals, who may or may not be professionally degreed, such as dieticians, physical trainers, exercise coaches, lifestyle coaches, meditation coaches, all of whom may be part of a holistic program of treatment for diabetics and pre-diabetics.

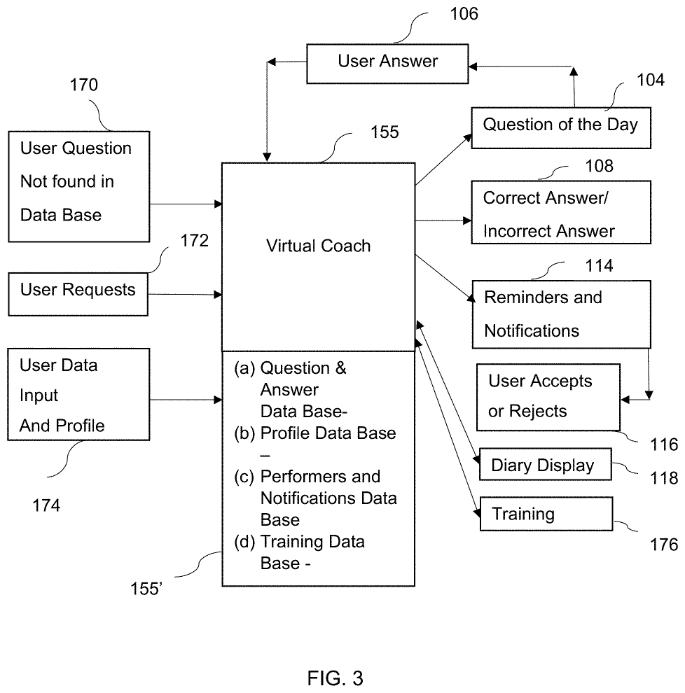

Schematic interconnections of an embodiment of the virtual coach are shown in FIG. 3, which illustrates an embodiment of user interactions and feedback with the system of FIG. 1.

In an embodiment, an artificial intelligence aspect of the virtual coach component 155 includes a question and answer data base 155(a), a user profile data base 155(b), a reminders and notifications data base 155(c) and a training data base 155(d). As defined herein, a user includes at least a subject intended to benefit by the system 100 for monitoring or delaying or preventing, or combinations thereof, the onset of Type 2 diabetes or reducing blood sugar to a normal level, who is part of a controlled participant network, or a system administrator or network coordinator for the controlled participant network. From the user device 140 of FIG. 1, a user, a user inputs a user profile and data input 101 to the virtual coach 155 wherein the user profile and data input 174 are stored in the user profile data base 155(b). From the user device 155, the user may input a user request 172 such as a question for which a response is anticipated from the question and answer data base 155(a).

If a question which the user is requesting an answer for is not found in the question and answer data base 155(a), the user my request via user interface 140 a question not found (170,) in the question and answer data base 155(a).

A user request 172 may also include a request for training wherein the virtual coach 155 displays a training user interface 176. As part of the training, the virtual coach 155 may pose to the user a question of the day user interface 104 to which the user provides a user answer at user interface 106. The virtual coach 102 indicates to the user via user interface 108 whether the user has provided a correct answer or an incorrect answer.

As part of reminders and notifications data base 155(c), the virtual coach provides a reminders and notifications user interface 114 which via user interface 116 the user accepts or rejects. Also as part of the reminders and notifications data base 155(c) and also the user profile data base 155(b), the virtual coach 155 provides a diary display user interface 118.

Based on insurance derived incentives to encourage use, compliance tools may be useful as part of the Module to monitor user compliance. Specific scores that are automatically tracked by the Type 2 Diabetes Prevention system 100 in product format include:
Diary entry compliance (diary entries made divided by diary entries requested)
Weight loss compliance (calculated weight loss compared to entered weight loss)
Verified weight compliance (comparison of entered weight loss compared to weight loss obtained from the user's physician)
Verified blood sugar reduced to normal (user's physician certification that user A1C is reduced to below 5.7)

Automated provisions are made such that the user is always aware of his or her compliance status (diary entry and weight loss compliance). Verified response (weight and blood sugar level) is obtained through a request by the user to his/her physician for release of medical information to the system administrator.

The diary entry compliance and weight compliance may be tracked by the system 100 and made apparent to the user but only made available to the system administrator upon request for incentive payment by the user. The verified weight and blood sugar score may be obtained by the user requesting these certifications from the physician and that the physician sends these values/scores to the system administrator. Based on these monitors, the insurance carrier may decide how they would like to tailor their own incentive program.

The virtual coach may be a neural network that learns behaviors of the user, and can anticipate or report on various behaviors. For example, a neural network may track exercise behavior over time, and learn to remind the user 30 minutes before a typical exercise time to do their exercise. Similarly, a neural network can report following an exercise session on statistics comparing a completed exercise session to past sessions. If the user is trying, for example, to increase their aerobic endurance, a neural network can report immediately that the user is on track and give a specific percentage, comparing the session to average of past sessions.

### Example

In an exemplary embodiment, a clinical study was conducted in ten patients having HbA1c of 5.7-6.4, classified as prediabetes. The patients were given a smartphone application (an “app”), called the “Type II Diabetes Prevention Module.” The Module is patterned after the NIH Diabetes Prevention Program Study (6) with a number of enhancements.

The Type II Diabetes Prevention Module is a secure cloud-based system which includes a web application focused on user education for diabetes prevention and a companion mobile (Android and iPhone) application that provides an electronic diary and virtual coach.

The Type II Diabetes Prevention Module incorporates material from the Centers for Disease Control (CDC) “Diabetes Prevention Recognition Program” (19). This material was used to produce educational material for the Module by producing slide presentations with audio and multiple-choice questions that must be properly completed by the user to be credited with completing each educational session. The user can select “Educational Sessions” to view any of 28 slide show sessions with audio. An “Educational Sessions Completed” option shows which sessions the user has reviewed, completed action plans for and passed the multiple-choice test and “Actions Plans Filed” to review goals and ways to cope saved by the user for each session completed.

Prior research has shown that reminders are associated with more effective implementation of intention and leads to positive goal-oriented behavior change. For example, the Type II Diabetes Prevention Module uses a virtual coach to remind the user to eat properly at each meal, to enter foods consumed into their diary, to exercise, to enter exercise completed into their diary, to complete educational sessions, to enter their weight into their diary, and to schedule appointments with their doctor. User inputs (dietary, exercise, education, and weight) are used to provide automated daily and weekly performance summaries as well as customized daily and weekly performance—based advice. The virtual coach is in constant contact with the user, providing more than 65 notifications each week to help keep the user focused on blood sugar control. A sample of the notification process from the virtual coach is shown in FIGS. 4A-C and the main Options screen of the mobile application is shown in FIG. 5 with tabs including Post Each Meal, Post Exercise, Post Weight, Monitor Exercise, Daily Summary, Weekly Summary, Apply for Incentives, Education, Compliance Status, and Logout.

In an embodiment, the inventive system will generate various messages on a predetermined schedule, or a dynamically determined basis. When a message is produced, a notification icon appears on the notification bar on the top of the phone screen, as shown in FIG. 4A. This icon indicates that the app wants to attract the attention of the user. FIG. 4B shows a notification screen after a user pulls the top notification bar down. The exemplary notification here is a reminder for an AM snack. If the user touches the notification, the screen in FIG. 4C appears, giving users the option to enter relevant information about the snack. A similar screen to 4C will appear for other user entry items, such as exercise and completion of educational programs.

In an aspect, the Module may provide the daily and weekly summaries to the user. Samples are shown in FIGS. 6A-C. FIG. 6A shows a daily summary provides the number of servings of each food group consumed, total calories consumed, total calories burned, and the projected weight loss for the week. FIGS. 6A and 6B show a two-page weekly summary with a graph of weight loss, current weight, weight last week, total weight loss, minutes of exercise for the week, and exercise calories burned for the week.

Physician engagement with the patient has been shown to be independently associated with significant weight loss and positively impact patient's behavior to engage in lifestyle changes including diet and weight loss. To facilitate this, the Module may include a notification dashboard that allows the user's physician to review user's progress and send notifications of encouragement. The dashboard may also be employed by a coach to keep each user with prediabetes on track to meet weight loss goals. The dashboard may summarize the following information for each user: weeks using the Module, educational sessions completed, % total compliance (percentage of dietary, exercise, and weight notification requests responded to), pounds lost, % weight compliance (percentage of weight notification requests responded to), minutes of activity entered, and % activity compliance (percentage of physical activity requests responded to).

In an embodiment, the user's physician or coach can review the dashboard and, based on user progress, select a notification to send from a drop-down, or provide a customized notification. The resultant notification is sent to the user.

The strength of the physician-patient relationship appears to allow many people with prediabetes to skip or progress rapidly through one or more behavioral steps in the process of lifestyle modification using the physician-supported cloud-based Module. The number of failures can most likely be minimized through a program that provides ample support and is easy to follow as described herein. Preventive technology that is directly associated with and supported by the physician may allow rapid advancement through behavioral stages and significantly improve preventive medicine program performance.

In an embodiment, the Module includes an educational component. In the Module as developed, the CDC educational program was incorporated. The CDC educational program consists of 26 educational PDFs. These PDFs were converted to PowerPoint-like slide shows with audio and incorporated into the Module web app. Two additional sessions were added. The first trains the user on the Module web app, while the second trains the user on the mobile app.

In an embodiment, a virtual coaching component is provided. In an embodiment, a human coach supplements the virtual coaching and provides supervision and personalized feedback to the users. The bulk of coaching may be provided by the virtual coach that is part of the cloud-based Module. Human coaching was periodically (approximately twice each week) made to each user based on review of the user's data on the dashboard. Users were also encouraged to text or call the coach with any issues or questions. The number of communications by the user to the coach via phone was small.

For the educational sessions, CDC recognition is based on delivering the program for at least 9 months to five or more participants with each completing three or more sessions within 6 months. Sixty percent of participants must complete nine or more sessions by month 6. In addition, 60% of participants must complete three additional sessions in months 7 to 12. Finally, the average participant weight loss must be >5%.

In an embodiment, a website may also be employed for registration and onboarding of patients with diabetes at the physician's office at the time of diagnosis. In total, 10 separate information screens were collected in the process. Starting in January 2018, patients with prediabetes were offered the Module for use as they were diagnosed. Enrollment was done in the family practice office of the co-author located in Middle Island, N.Y. Over an approximate 2-month period, 10 patients with prediabetes were offered and accepted Module use. The goal was to recruit sufficient users to obtain CDC recognition. After recruitment of 10 users, 8 users were compliant, so recruitment was stopped. Shortly thereafter, two more users withdrew leaving six users who completed the study.

Ten patients were enrolled in the study and six completed the study. Of these six patients, five were female and one was male. The age of the group ranged from 44 to 63 years with the average and standard deviation of 53.1±9.1 years. Four were white, one black/African American, and one Asian. The study participants recorded dietary, exercise, and weight data, and have lost weight as summarized in Table I.

TABLE 1
Data of the six patients with prediabetes that have continued Module use for approximately 6 months.

| Active | Initial | | Educational | Dietary | Pounds | Weight | Minutes | Activity | % Weight | Activity |
|---|---|---|---|---|---|---|---|---|---|---|
| user | weight | Weeks | sessions | compliance (%) | lost | compliance (%) | of activity | compliance (%) | lost | minutes/week |
| A | 119 | 21 | 7 | 60 | −14 | 90 | 7009 | 80 | −11.8 | 334 |
| B | 200 | 21 | 12 | 88 | −19 | 90 | 2556 | 86 | −9.5 | 122 |
| C | 223 | 20 | 13 | 79 | −14 | 90 | 3585 | 52 | −6.3 | 179 |
| D | 165 | 12 | 13 | 89 | −20 | 100 | 3524 | 95 | −12.1 | 294 |
| E | 232 | 18 | 15 | 86 | −19 | 83 | 3210 | 73 | −8.2 | 178 |
| F | 112 | 27 | 16 | 69 | −7 | 92 | 2937 | 70 | −6.3 | 109 |
| Average | | | 13 | | | | | | −9.0 | 192 |

All six met the CDC-specified weight loss target of 5% of their body weight. Weight loss accuracy was verified for each user in the second quarter of use by the physician. Since 6 of the 10 users have met the weight loss target, the success rate of the Module is approximately 60%. The average number of educational sessions completed for these six users was approximately 13 in total, 11 of which were CDC sessions. The average user weight loss was approximately 9.0% and the average minutes of physical activity was 192 min per week.

Percent compliance in Table I provides the percent of the time the user responded to requests from the virtual coach to enter meal data (six times each day), exercise data (daily), and weight data (weekly). User compliance with requests from the virtual coach for dietary data input ranged from 59% to 87%, while physical activity ranged from 52% to 93% and weight data ranged from 83% to 100%. Compliance was quite good for all users and there is a correlation of weight loss with compliance. For example, the user with the highest percent of weight loss also had the highest compliance rates, while the users with the lowest percent of weight loss had lower compliance rates.

While physician support of lifestyle modification is shown here to greatly impact success, other support mechanisms may provide similar results. Another possible driver of lifestyle modification success may include incentives rather than the physician support. The American Diabetes Association reports that the insurance industry is expected to save $9600 in direct medical costs each year that diabetes is prevented for a single patient, this suggests employers and policy makers will be motivated to support an incentive-based program. Incentives have been shown to motivate participation in wellness programs. For example, employees of a large corporation were offered an online physical activity program that initially saw only 13% participation by eligible staff. The same program was offered a year later with a $150 cash incentive. The incentive led to 53% participation with a remarkable 74% of those completing the program and qualifying for the incentive.

Many people with prediabetes, on their own, decide to heed their physician's advice to lose weight, exercise, and join a gym. The availability of the Module to these gym members through their coach at the gym may also assure improved success since the user may see the coach at the gym several times each week. For the gym, the module has potential important benefits. The gym may obtain CDC Recognition Status, offer this additional service, and also seek reimbursement for diabetes prevention services from insurance carriers. Since the diabetes prevention program is a 1- or 2-year process, participant membership would also be long term.

### Implementation

In order to implement the Module as described above, a system, may be provided with a memory that stores computer executable components. The system may include a processor, operably coupled to the memory, and that executes computer executable components stored in the memory, wherein the computer executable components comprise components tracking exercise, food consumption, body weight, blood sugar measurements, and blood A1C data, or a combination thereof of a user diagnosed with diabetes or pre-diabetes. The system may include a virtual coaching component that determines a set of optimized dietary or optimized lifestyle goal scores, dietary goal scores, and optimized exercise scores, based on algorithms employing data on exercise, food consumption, body weight, blood sugar measurements, and blood A1C data, or a combination thereof of the user. A database, operably coupled to the processor, stores the optimized lifestyle, dietary, and exercise scores. The system may determine scores that are used to by the virtual coaching component to make real-time specific recommendations of diet, exercise, and lifestyle elements calculated to reduce blood sugar in the user. In an embodiment, the system runs on a computer. In an embodiment, the computer is a smartphone or tablet (i.e., Apple iOS or Android). In an embodiment, the computer is a server running one or processes (run-time processes) that serve web pages to a standard web-browser, such as Google Chrome, Firefox, or Microsoft Edge.

In an embodiment, the system may provide real-time specific recommendations of diet, exercise, and lifestyle elements, which are presented on a user-interface on a smartphone or web app.

In an embodiment, the system the system may provide real-time specific recommendations of diet, exercise, and lifestyle elements to the user at least three times per day.

In an embodiment, the system may include one or more databases that store various user data, such as exercise and health-related data. This allows data (for example, exercise goals) to be measured over time and presented as reports to users. These reports can be powerful motivational tools for users.

In an embodiment, a user interface is provided for a user diagnosed with diabetes or pre-diabetes. The user interface provides a means for the user to interact with computer application that includes a memory that stores computer executable components. The computer application may also include a processor, operably coupled to the memory, and that executes computer executable components stored in the memory, wherein the computer executable components comprise components tracking exercise, food consumption, body weight, blood sugar measurements, and blood A1C data, or a combination thereof of a user diagnosed with diabetes or pre-diabetes. The computer application may also include a virtual coaching component that determines a set of optimized dietary or optimized exercise scores, or both, based on algorithms employing data on exercise, food consumption, body weight, blood sugar measurements, and blood A1C data, or a combination thereof of the user. The scores may be used to by the virtual coaching component to make real-time specific recommendations of diet, exercise, and lifestyle elements calculated to reduce blood sugar in the user.

The artificial intelligence aspect of the virtual coach may rely on various algorithms that may be employed in the system for computing relevant information and calculating scores used to generate messages and user instructions for the virtual coach, supervisors (human coaches), and physicians.

Many calculators may be used to “coach” the user. Many of the inputs for these calculations come as a result of user response to notifications as described above. In addition, user data collected upon acquiring the module (on boarding process) is used:
user weight
user height
user gender
user age
user life activity level (1-4)

Some exemplary messages that may be generated by the Virtual coach are shown below. These messages are algorithmically produced from scores obtained from various memory components. The following text responses will be made by the virtual coach after the daily summary:

| Response | Condition |
|---|---|
| Please complete your diary! | User has not completed entry of |
| | meal/exercise data |
| Congratulations! You met | User meets food targets such |
| your dietary target. | that calories consumed do not |
| | exceed their target by |
| | more than 10%. |
| A good day! You nearly | User exceeded target calories |
| met your dietary target. | between 10 and 25%. |
| Try to avoid sweets. They spike | User consumed one or more |
| your blood sugar. | servings of sweets. |
| Little progress on weight loss | User exceeds target calories |
| today. Make tomorrow a better day | by more than 25% |
| by eating healthy! | |

Other exemplary components are shown below. Inputs for the calculations of calories consumed 2520, calories burned 2530 and projected weight loss 2560 are derived from an initial questionnaire and the user's diary. The details of each calculation are given below:

In an embodiment, distance walked and walking speed data may be collected.

Inputs: gender, steps taken (cell phone step counter), walking time (cell phone step counter in minutes)

Stride factor=0.413 for females and 0.415 for males

Distance walked (miles)={[stride factor×height (inches)/12]×steps taken}/5,280

Speed (mph)=[distance walked(miles)/walking time (min)]/60

In an embodiment, calories burned in walking may be computed.

Inputs: weight (lb), miles walked (computed above)

Calories burned=weight×miles walked×0.3

Calories burned, as determined with the step counter may be automatically stored in the user exercise diary (i.e., database). Alternatively, the user can simply input minutes for a particular exercise into their diary using the screen below. The calories burned through exercise can then be calculated using published values of calories burned per minute for that exercise corrected for user weight.

Calorie consumption components may be incorporated into the Module. The user may input the number of servings of starches, vegetables, fruit, milk, meat/meat substitutes, fats and sweets into their diary at each meal. This may be done by the user through the user notification process (FIGS. 4A-C). Alternatively, this data may be entered using the options screen (FIG. 5). With either option, the user is presented with a user input screen, exemplified in FIG. 4C.

At the end of the day, the total number of calories consumed can be calculated from the total servings data as follows: Calories consumed=starch servings×80+vegetable servings×25+fruit servings×60+milk servings×90+meat/meat substitute servings×75+fat servings×45+sweet servings×80.

The system may compute calories required per day (to maintain weight). Inputs from on boarding are used to calculate calories required per day including: age, weight, height, gender and activity level (1-4).

The correction for gender is to add 5 for males and subtract 161 for females

The correction for activity level is to multiply by 1 for sedentary, multiply by 1.2 for low activity, multiply by 1.27 for active and multiply by 1.45 for very active.

Calories required per day=[9.9×weight (kg)+2.25×height (cm)+4.92×age in years+gender correction]×activity correction

In an embodiment, the system may compute projected weight loss per week (from one day's data)

The following output from the calculators are used: calories required per day, calories burned, calories consumed.

Projected weight loss=7×(calories required+calories burned−calories consumed)/3500

This data is presented to the user in the screen below at the end of each day.

Body mass index (BMI)

Inputs: weight (lb) and height (inches)BMI=[weight/(height2)]×703

Body mass index (BMI)

Inputs: weight (lb) and height (inches)BMI=[weight/(height2)]×703

Distance walked and speed

Inputs: gender, steps taken (cell phone step counter), walking time (cell phone step counter in minutes)

Stride factor=0.413 for females and 0.415 for males

Distance walked (miles)={[stride factor×height (inches)/12]×steps taken}/5,280Speed (mph)=[distance walked (miles)/walking time (min)]/60

Calories burned in walking

Inputs: weight (lb), miles walked (computed above) Calories burned=weight×miles walked×0.654

Calories consumed

Inputs: servings of starch, vegetables, fruit, milk, meat/meat substitutes, fats and sweets (from subject diary)

Calories consumed=starch servings×80+vegetable servings×25+fruit servings×60+milk servings×90+meat/meat substitute servings×75+fat servings×45+sweet servings×80.

Calories required per day

Inputs from questionnaire: age, weight, height, gender and activity level (1-4)

Correction for gender is to add 5 for males and subtract 161 for females

Correction for activity level is to multiply by 1 for sedentary, multiply by 1.2 for low activity, multiply by 1.27 for active and multiply by 1.45 for very active.

Calories required per day=[9.9×weight (kg)+2.25×height (cm)+4.92×age in years+gender correction]×activity correction.

Projected weight loss per week (from one day's data).

Inputs from calculators are the following: calories required per day, calories burned, calories consumed.

Projected weight loss=7×(calories required+calories burned−calories consumed)/3500

Using these calculations, the artificial intelligence aspect of the virtual coach can generate notifications, messages, and reports that help the user understand their condition and take steps calculated to reduce blood sugar, lower body weight, increase exercise, and make healthy lifestyle choices.

### REFERENCES

1. American Diabetes Association. Classification and diagnosis of diabetes: standards of medical care in diabetes-2018. Diabetes Care 2018; 41(Suppl. 1): S13-S27.
2. Ali, M K, Bullard, K M, Saydah, S. Cardiovascular and renal burdens of prediabetes in the USA: analysis of data from serial cross-sectional surveys, 1988-2014. Lancet Diabetes Endocrinol 2018; 6(5): 392-403.
3. Centers for Disease Control and Prevention. National diabetes statistics report, 2017. Atlanta, Ga.: Centers for Disease Control and Prevention, US Department of Health and Human Services, 2017.
4. Dall, T M, Yang, W, Halder, P. The economic burden of elevated blood glucose levels in 2012: diagnosed and undiagnosed diabetes, gestational diabetes mellitus, and prediabetes. Diabetes Care 2014; 37(12): 3172-3179.
5. Tuomilehto, J, Lindstrom, J, Eriksson, J G. Prevention of type 2 diabetes mellitus by changes in lifestyle among subjects with impaired glucose tolerance. N Engl J Med 2001; 344(18): 1343-1350.
6. Knowler, W C, Barrett-Connor, E, Fowler, S E. Reduction in the incidence of type 2 diabetes with lifestyle intervention or metformin. N Engl J Med 2002; 346(6): 393-403.
7. Schellenberg, E S, Dryden, D M, Vandermeer, B. Lifestyle interventions for patients with and at risk for type 2 diabetes: a systematic review and meta-analysis. Ann Intern Med 2013; 159(8): 543-551.
8. Johnson, L N, Melton, S T. Perceived benefits and barriers to the diabetes prevention program, http://theplaidjournal.com/index.php/CoM/article/view/65/49
9. Pew Research Center. Mobile phone ownership, 2017, http://www.pewinternet.org/chart/mobile-phone-ownership/
10. Schippers, M, Adam, P C, Smolenski, D J. A meta-analysis of overall effects of weight loss interventions delivered via mobile phones and effect size differences according to delivery mode, personal contact, and intervention intensity and duration. Obes Rev 2017; 18(4): 450-459.
11. Fukuoka, Y, Gay, C L, Joiner, K L. A novel diabetes prevention intervention using a mobile app: a randomized controlled trial with overweight adults at risk. Am J Prey Med 2015; 49(2): 223-237.
12. Moin, T, Ertl, K, Schneider, J. Women veterans' experience with a web-based diabetes prevention program: a qualitative study to inform future practice. J Med Internet Res 2015; 17(5): e127.
13. Block, G, Azar, K M, Romanelli, R J. Diabetes prevention and weight loss with a fully automated behavioral intervention by email, web, and mobile phone: a randomized controlled trial among persons with prediabetes. J Med Internet Res 2015; 17(10): e240.
14. Allen, J K, Stephens, J, Dennison Himmelfarb, C R. Randomized controlled pilot study testing use of smartphone technology for obesity treatment. J Obes 2013; 2013: 151597.
15. Tate, D F, Finkelstein, E A, Khavjou, O. Cost effectiveness of internet interventions: review and recommendations. Ann Behav Med 2009; 38(1): 40-45.
16. Diabetes Prevention Program Research Group. The 10-year cost-effectiveness of lifestyle intervention or metformin for diabetes prevention: an intent-to-treat analysis of the DPP/DPPOS. Diabetes Care 2012; 35(4): 723-730.
17. Smith, K J, Kuo, S, Zgibor, J C. Cost effectiveness of an internet-delivered lifestyle intervention in primary care patients with high cardiovascular risk. Prey Med 2016; 87: 103-109.
18. Grock, S, Ku, J H, Kim, J. A review of technology-assisted interventions for diabetes prevention. Curr Diab Rep 2017; 17(11): 107.
19. https://www.cdc.gov/diabetes/prevention/pdf/dprp-standards.pdf

1-33. (canceled)

34. A system, comprising
a. a memory that stores computer executable components;
b. a processor, operably coupled to the memory, and that executes computer executable components stored in the memory, wherein the computer executable components comprise components tracking exercise, food consumption, body weight, blood sugar measurements, and blood A1C data, or a combination thereof of a user diagnosed with diabetes or pre-diabetes; and
c. a virtual coaching component that determines a set of optimized lifestyle goal scores, dietary goal scores, and optimized exercise scores, based on algorithms employing data on exercise, food consumption, body weight, blood sugar measurements, and blood A1C data, or a combination thereof of the user; and
d. wherein a database, operably coupled to the processor, stores the optimized lifestyle, dietary, and exercise scores; and
e. wherein the scores are used to by the virtual coaching component to make real-time specific recommendations of diet, exercise, and lifestyle elements calculated to reduce blood sugar in the user.

35. The system of claim [0013], wherein the real-time specific recommendations of diet, exercise, and lifestyle elements are presented on a user-interface on a smartphone or web app.

36. The system of claim [0013], wherein the real-time specific recommendations of diet, exercise, and lifestyle elements provided to the user at least three times per day.
